(19) United States
(12) Patent Application Publication
(10) Pub. No.: US 2015/0011821 A1
(43) Pub. Date: Jan. 8, 2015

(54) MECHANICAL MYOCARDIAL RESTRAINT DEVICE

(71) Applicant: The Trustees of The University of Pennsylania, Philadelphia, PA (US)

(72) Inventors: Robert C. Gorman, Lower Gwynedd, PA (US); Matthew J. Gillespie, Bryn Mawr, PA (US); Joseph H. Gorman, Lower Gwynedd, PA (US)

(21) Appl. No.: 14/366,524

(22) PCT Filed: Dec. 21, 2012
(86) PCT No.: PCT/US2012/071229

§ 371 Date: Jun. 18, 2014

Related U.S. Application Data
(60) Provisional application No. 61/578,492, filed on Dec. 21, 2011.

(51) Int. Cl. A61F 2/24

(52) U.S. Cl. CPC A61F 2/2481; A61F 2002/2484; A61F 2220/0008; A61F 2230/0091; A61F 2250/0003
USPC 600/37

(57) ABSTRACT

A transvenously deployed myocardial restraint device provides mechanical restraint of a dilated left ventricle and improves heart function. The device includes a delivery wire with a restraining coil that is straightened for percutaneous delivery to the heart and recoiled once positioned in a pericardial space adjacent a ventricular heart wall. Alternatively, a balloon on the end of the delivery wire is delivered similarly to the coil and is then inflated in the pericardial space to restrain the dilated portion of the left ventricle. A trailing end of the coil forms a tether that is anchored to the left ventricular free wall and to the ventricular septum by an intraventricular septal anchor and connected by the tether through the ventricular septum using the same delivery system.

### CROSS-REFERENCE TO RELATED APPLICATIONS

The present application claim priority to U.S. Provisional Patent Application No. 61/578,492, filed Dec. 21, 2011. The content of that application is herein incorporated by reference in its entirety.

### TECHNICAL FIELD

The invention relates to a restraint device for improving heart function by mechanically restraining a dilated left ventricle as a result of myocardial infarction (MI) or other heart diseases and injuries causing left ventricular remodeling.

### BACKGROUND

Myocardial infarction (MI) initiates a cascade of events that eventually culminates in left ventricular (LV) dilation, depressed function, dyskinesis, heart failure, and eventually death. Surgical myocardial/epicardial restraint of the infarcted left LV has been shown to attenuate and reverse LV remodeling, resulting in improved myocardial mechanics and function. A minimally invasive, percutaneously deployed myocardial restraint device is desired to address these problems. Such a device would potentially benefit millions of MI survivors.

Patients suffer from ischemic mitral valve regurgitation (IMR) as a result of left ventricular dilatation, often caused by MI. This population of patients in the United States alone is estimated to be 1.2 to 2.1 million patients, with approximately 425,000 patients having moderate or severe IMR with heart failure. IMR results from left ventricular (LV) distortions caused by a myocardial infarction (MI) or heart attack. Patients with this disease survive their heart attack but the resulting injury causes the ventricle to dilate and fail over months and years. In many cases, this congestive heart failure (CHF) is worsened by IMR. Patients with CHF and IMR can become extremely sick and be very hard to manage medically. Most clinicians agree that a competent mitral valve would make the management of these patients much more straight-forward and cost effective. The restraint device proposed herein further offers a minimally invasive way to reduce the septal-lateral dimension of the LV, potentially improving mitral valve function.

The inventors are not aware of any comparable, commercially available technologies for restraining a myocardial wall as proposed by the invention. There are, however, a number of percutaneous mitral valve repair devices that have been developed to exploit the proximity of the coronary sinus to the mitral valve annulus to perform some type of “annuloplasty” to limit mitral regurgitation. The basic premise behind such devices is to place a device in the coronary sinus that will shrink the valve orifice and thus decrease mitral regurgitation. None of these techniques have shown reproducible efficacy in human trials and none has proposed to restrain the dilated LV.

### SUMMARY

The inventors have addressed the above needs in the art by developing a set of devices that may be deployed using a completely percutaneous transvenous approach or percutaneous transvenous approach in combination with a minimally invasive (small incision) surgical approach to provide mechanical restraint of a dilated left ventricle. In exemplary embodiments, the device includes a delivery wire that is straightened for percutaneous transvenous delivery to the heart. Once in the heart the delivery wire is passed through the ventricular septum to gain access to the left ventricle (LV). It is then passed through the infarcted region of the LV and assumes its preformed coiled shape once positioned in a pericardial space adjacent an exterior left ventricular heart wall. The coiled wire is then pulled snugly against the infracted region and held in place by an intracardiac anchor that may be small inflatable balloons, pads of material, flexible wire weave mesh disks, etc. that also serve to provide hemostasis. A trailing end of the delivery wire forms a tether that is anchored to the ventricular septum by an additional pair of intracardiac pads, mesh disks or balloons deployed on both sides of the ventricular septum and connected by the tether through the ventricular septum. A coaxial locking mechanism secures the intracardic anchors to the tether. The distance between the ventricular septum and the ventricular heart wall where the restraining coiled wire is positioned is shortened by tightening the tether connecting the restraining coiled wire to the anchor. The restraining coiled wire is preferably preformed to be concave toward the ventricle so as to cup the ventricular heart wall when deployed. The restraining coiled wire could also be designed to be flat for placement on other portions of the left ventricle.

In an alternative embodiment, the restraining coiled wire on the end of the delivery wire is replaced by a balloon affixed to the distal end of the delivery wire. The balloon is also adapted for transvenous delivery to the pericardial space and adapted to be inflated in the pericardial space for restraint against the surface of the LV once positioned.

In other exemplary embodiments, a flexible metallic (nitinol) retention mesh is directed into the pericardial space via a sub-xiphoid incision or thoracotomy and the retention mesh is subsequently joined to a delivery wire that is placed transvenously traversing both the ventricular septum and left ventricular free wall at the area of infarction in the same manner as described above for the totally transvenous embodiments. The delivery wire is used to pull the restraining mesh against the LV freewall and the LV-ventricular septal dimension is reduced by anchoring the retention cable in both the septum and LV cavity side of the freewall. Alternatively, a balloon may be directed into the pericardial space via a sub-xiphoid incision or thoracotomy, inflated, and then joined to the transvenous delivery wire. These devices are anchored using a similar technique as that described above.

The invention also includes methods for a percutaneous transvenous delivery of the mechanical restraint device of the invention to the heart. For example, an exemplary delivery method of the invention includes:

introducing a vascular introducer sheath into a vein and advancing the introducer sheath into a right ventricular cavity of the heart to gain access to the ventricular septum;

puncturing the ventricular septum with a transeptal needle;

advancing the introducer sheath and a dilator over the transeptal needle into the left ventricular cavity;

puncturing the left ventricular wall at a desired site and advancing the introducer sheath into pericardial space;

removing the dilator and needle;

delivering a delivery wire via the introducer sheath into the pericardial space where the delivery wire recoils in the pericardial space to a preformed coil shape or a balloon on the distal end of the delivery wire is deployed in the pericardial space by a similar method;

pulling the recoiled restraining coiled wire or balloon snug against an epicardial surface of the left ventricular wall;

withdrawing the introducer sheath while maintaining traction on a trailing end of the delivery wire as a tether;

anchoring the tether to a series (usually 2) of intracardiac anchors (small balloon, wire weave meshes, or material pad) placed on either side of the ventricular septum as the introducer sheath is withdrawn from the left ventricle into the right ventricle;

tightening the tether as desired; and

locking the septal anchors to the tether.

In the exemplary method, the coiled wire or balloon is preferably preformed to be concaved toward the ventricle to cup the ventricular heart wall when deployed. The coiled wire or balloon could also be designed to be flat for placement on other portions of the left ventricle. The desired site in the left ventricular wall is preferably at a location of left ventricular dilation or other injury such as that caused by myocardial infarction.

Alternatively, the method of deploying the myocardial restraint device in the heart may include replacing the step of recoiling the restraining coil or deploying the balloon into the pericardial space with the step of delivering a coiled wire, wire mesh or balloon restraining device via a minimally invasive sub-xiphoid incision or thoracotomy into the pericardial space where it is connected to a delivery wire which has been previously placed through the infarct into the pericardial space (as described above in the completely transvenous approach). Once this connection is made (using a screw, magnet, balloon, or other similar mechanisms) the delivery wire is pulled snug against the infarct and anchored and tethered to the ventricular septum as previously described for the completely transvenous approach.

Other anchoring devices and anchoring methods also may be used in a manner consistent with the following description.

### BRIEF DESCRIPTION OF THE DRAWINGS

The various novel aspects of the invention will be apparent from the following detailed description of the invention taken in conjunction with the accompanying drawings, of which:

FIG. 1 illustrates a heart with a deployed myocardial restraint device in accordance with a first exemplary embodiment of the invention.

FIG. 2 illustrates a perspective view of the heart with the deployed myocardial restraint device of the embodiment of FIG. 1.

FIG. 3 illustrates a heart with a deployed myocardial restraint device in accordance with a second exemplary embodiment of the invention where the recoiled wire is replaced with an inflatable balloon.

FIG. 4 illustrates an alternative embodiment of the deployment of a myocardial restraint device where the transvenous retention cable is anchored by a separate flexible wire webbing, coil or mesh placed on the epicardial surface via a sub-xiphoid incision or thoracotomy.

FIG. 5 illustrates a further alternative embodiment of the deployment of a myocardial restraint device where the transvenous retention cable is anchored by a balloon separated from the tether that is placed on the epicardial surface via a sub-xiphoid incision or thoracotomy and inflated to anchor the tether in place through the wall of the left ventricle.

### DETAILED DESCRIPTION OF ILLUSTRATIVE EMBODIMENTS

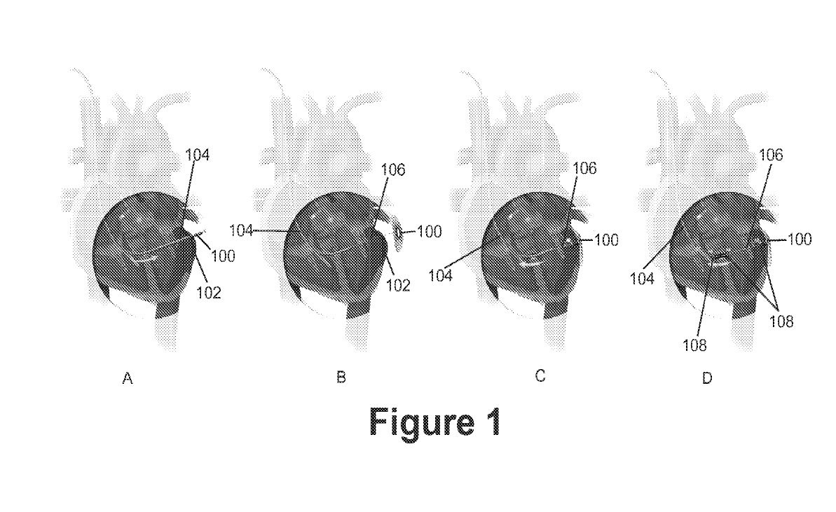

The invention will be described in detail below with reference to FIGS. 1-5. Those skilled in the art will appreciate that the description given herein with respect to those figures is for exemplary purposes only and is not intended in any way to limit the scope of the invention. All questions regarding the scope of the invention may be resolved by referring to the appended claims.

### Overview

The inventors have developed approaches to post infarction left ventricular mechanical restraint. In exemplary embodiments, a myocardial restraint device is provided that is designed to tether the left ventricular freewall to the ventricular septum and includes 4 main components. First, a delivery wire is provided comprising a thick metal wire (nitinol or stainless steel) preformed into a broad, tight, spiral coil that is concave in shape so that it will effectively “cup” the left ventricular freewall when pulled against the epicardial surface. In an exemplary embodiment, the coiled wire is circular in shape and 5-10 cm in diameter. Conversely, a balloon may be deployed on the distal end of the delivery wire and advanced through the left ventricular freewall, and inflated to anchor the tether. Second, a tether is provided that is formed from the trailing end of the delivery wire to function as a restraining wire. Third, an intraventricular septal anchor is provided that may include a broad nitinol pad, wire weave meshes, or balloons on both the right and left ventricular sides of the septum that is connected by a central stalk running through the septal muscle. Fourth, a locking mechanism such as a coaxial locking mechanism is threaded over the tether to hold the septal anchor in place. Several embodiments of such a myocardial restraint device will be described below.

In each embodiment described below, the myocardial restraint device is placed at a location to be restrained (e.g., at location of infarct) strictly transvenously or via a combination of transvenous and minimally invasive surgery. For example, the wire can be placed transvenously through the septum and then through the left ventricle free wall and the coiled portion uncoiled to form a restraining disc as an anchor in the pericardial space. Alternatively, the tether may be connected to a webbing/mesh restraining device advanced into place via a small sub-xiphoid incision or thoracotomy and connected to the tether in the pericardial space. On the other hand, a balloon or other restraining device may be advanced transvenously through the left ventricle free wall to anchor the tether in place, or a restraining balloon may be delivered via a small sub-xiphoid incision or thoracotomy, advanced into position, and then attached to the tether and inflated. Such myocardial constraint configurations are illustrated in the respective embodiments of FIGS. 1-5.

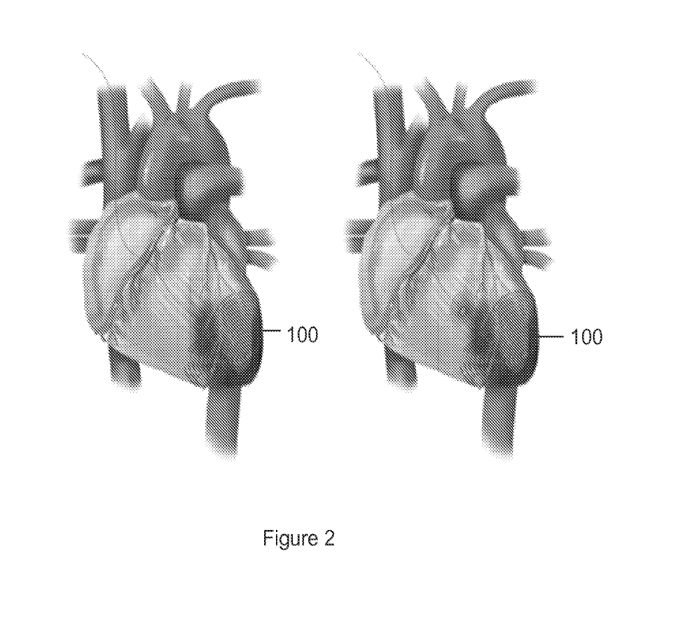

### Embodiment of FIGS. 1 and 2

A first exemplary embodiment of the myocardial restraint device of the invention is depicted in FIGS. 1A-1D and FIG. 2. FIGS. 1A-1D illustrate sequential deployment of a first embodiment of the myocardial restraint device, while FIG. 2 illustrates a perspective view of the deployed device on the epicardial surface of the left ventricle. As illustrated in FIG. 1A, the delivery wire 100 is advanced transvenously through the ventricular septum and the free left ventricular wall in the area of the infarct 102 using a suitable catheter 104. Once through the ventricular wall, the restraint device, in the form of a nitinol or metal delivery wire 100, assumes its preformed coiled shape as shown in FIG. 1B to anchor the tether 106, typically an extension of the delivery wire 100. The catheter 104 is then retracted as shown in FIG. 1C and the tether 106 is pulled taut from the proximal end of the delivery wire 100. The tether 106 is then deployed and anchored using a coaxial locking mechanism 108 that anchors the taut tether 106 to respective sides of the septum.

As illustrated in FIG. 1, the restraint device so configured is delivered via percutaneous transvenous access (e.g., jugular, subclavian, femoral vein[s]) in a stepwise manner as follows:
1) A vascular introducer sheath (e.g., 7 French) is inserted into the right jugular or subclavian vein (or femoral vein) and advanced into the right ventricular cavity.
2) The tip of the sheath is apposed to the ventricular septum under echocardiographic guidance, and the septum is punctured with a transeptal needle.
3) The vascular sheath and dilator are advanced over the needle into the left ventricular cavity.
4) The transeptal needle is then positioned for puncture of the left ventricular freewall/apex (the area of infarction or injury).
5) As in steps 3-4, the left ventricular freewall is punctured and the delivery sheath is advanced into the pericardial space (FIG. 1A). The inner dilator and needle are removed.
6) The ventricular restraint device is then delivered via the introducer sheath into the pericardial space as shown in FIG. 1B and then is pulled snug against the epicardial surface as shown in FIG. 1C. The coiled end of delivery wire 100 is straightened for delivery via the catheter 104 and then recoils to its nominal “circular” coiled shape once it is extruded through the catheter 104 and into the pericardial space (FIG. 1B). The coiled end of delivery wire 100 may be preformed to be concave toward the ventricle so that it will effectively “cup” the left ventricular freewall when pulled against the epicardial surface as shown in FIG. 2 or else the coiled end of the delivery wire 100 may be flat for placement on other portions of the left ventricle such as a posterior wall. The trailing end 106 of the delivery wire 100 serves as the “tether” for the “restraining coil” (FIG. 1C). This trailing also may promote hemostasis.
7) The introducer sheath is then withdrawn into the left ventricular cavity, keeping gentle traction on the “tether” portion 106 of the delivery wire 100 to minimize bleeding through the freewall puncture site (FIG. 1C).
8) The tether 106 is attached to an “anchor” 108 placed within the ventricular septum as the delivery sheath is withdrawn from the left ventricle and into the right ventricle (FIG. 1D). The anchoring device 108 may include a pair of nitinol pads, wire weave meshes, or balloons on both the right and left ventricular sides of the septum and inside of the LV wall, connected by a central stalk of the tether 106 running through the septal muscle (FIG. 1D).
9) The septal to freewall distance can be shortened by tightening the tether 106 that connects the delivery wire 100 to the septal anchor 108 as desired (FIG. 1D).
10) The anchor 108 is, for example, locked to the tether 106 using a coaxial slot and groove mechanism or pads, meshes, or balloons as described above. In an exemplary embodiment, the tether 106 extends through the middle of the anchor 108, which is straddling the ventricular septum. The tether 106 has a series of slots on the right ventricular side, which are slid into a locking groove on the right ventricular side of the anchor 108 once the septal-to-freewall dimension is determined by tension on the tether 106. The slot-in-grove lock maintains the set tension on the tether crossing the left ventricular cavity, thus maintaining the septal-to-freewall distance and the desired amount of restraint.

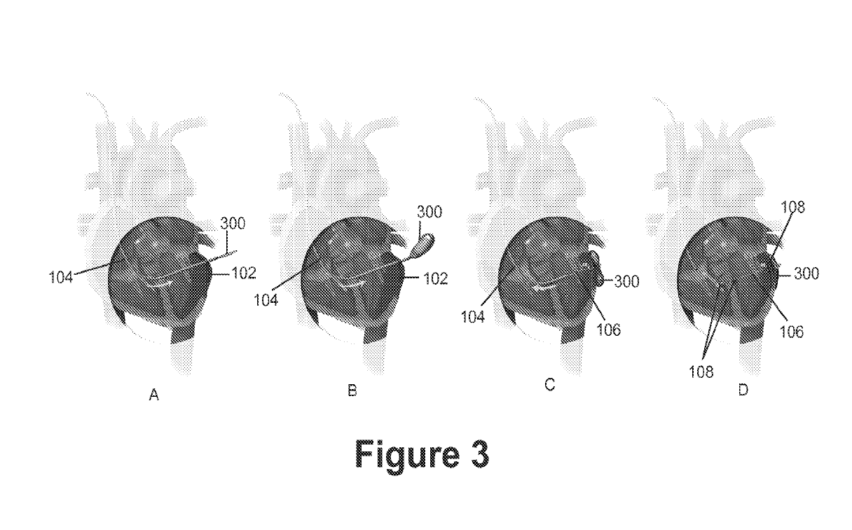

### Embodiment of FIG. 3

FIG. 3 illustrates a heart with a deployed myocardial restraint device in accordance with a second exemplary embodiment of the invention where the recoiled wire of the embodiment of FIG. 1 is replaced with an inflated balloon. In this embodiment, the balloon 300 is connected to a distal end of the delivery wire 100 in its thin deflated state and is passed transvenously through the ventricular septum and the free left ventricular wall as in the first embodiment (FIG. 3A). Once in the pericardial space, the balloon is inflated (FIG. 3B) and pulled against the left ventricular wall by pulling a proximal end of the delivery wire 100 (FIG. 3C). The balloon 300 may be preformed to be concave toward the ventricle so that it will effectively “cup” the left ventricular freewall when deployed or else the balloon 300 may be flat for placement on other portions of the left ventricle such as a posterior wall. As in the first embodiment, the trailing end 106 of the delivery wire 100 connected to the balloon 300 serves as the tether 106 for the restraint device 100. Once the balloon 300 has been deployed, the catheter 104 is then retracted and the tether 106 is pulled taut from the proximal end of the delivery wire 100. As in the first embodiment, the restraint device is then deployed and anchored using a coaxial locking mechanism and anchors 108 that anchor the taut tether 106 to respective sides of the septum (FIG. 3D).

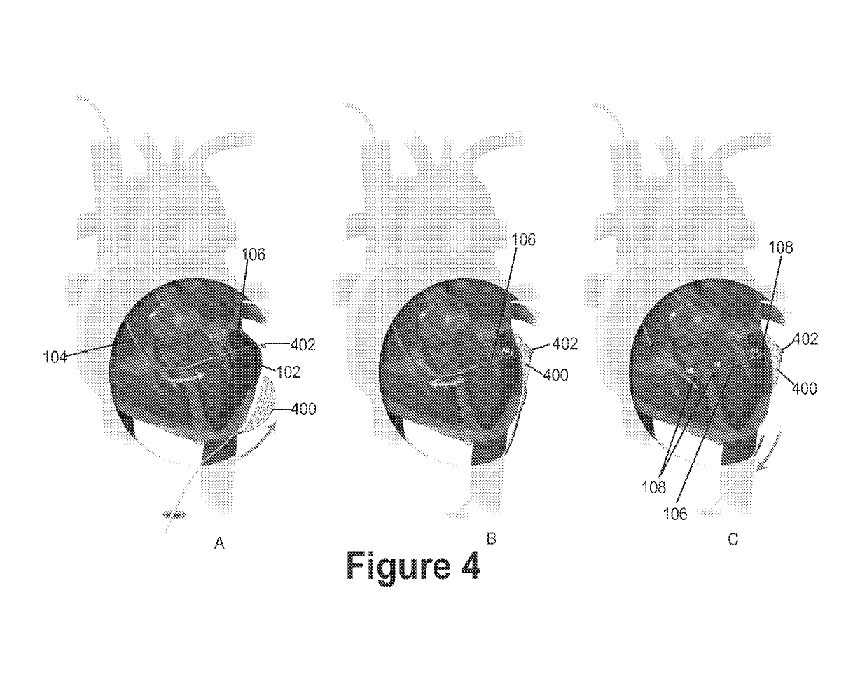

### Embodiment of FIG. 4

FIG. 4 illustrates an alternative embodiment of the deployment of a myocardial restraint device where the anchor is a separate coil or webbing that is introduced via a minimally invasive sub-xiphoid incision or thoracotomy and advanced into the pericardial space to anchor the tether 106 in place through the wall of the left ventricle. In this embodiment, the retention coil or mesh 400 blossoms into its predetermined shape once deployed in the pericardial space and is joined to the delivery wire 100 that has been advanced transvenously traversing both the ventricular septum and left ventricular free wall at the area of infarction 102 as shown in FIG. 4B. The delivery wire 100 functions as a retention cable having an end such as a screw, magnet, or balloon 402 that is deployed through a cavity or hole in the retention coil or mesh 400 and is used to pull the retention coil or mesh 400 against the LV freewall. As in the embodiments of FIGS. 1-3, the LV ventricular-septal dimension is reduced by anchoring the retention cable in both the septum and LV cavity side of the freewall (FIG. 4C) using coaxial locking mechanism 108.

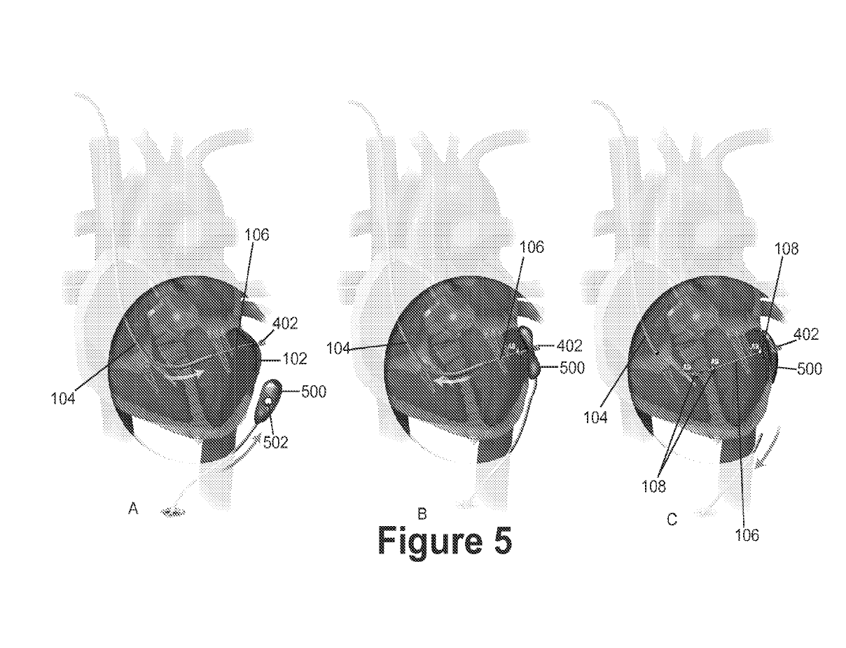

### Embodiment of FIG. 5

FIG. 5 illustrates a further alternative embodiment of the deployment of a myocardial restraint device where the restraint device includes a balloon 500 introduced via a minimally invasive sub-xiphoid incision or thoracotomy and separate from the tether 206 that is advanced transvenously through the ventricular septum. The delivery wire 100 is joined to the balloon 500 as shown in FIG. 5B. In an exemplary embodiment, the balloon 500 has a cavity or hole 502 that accepts the screw, magnet, or balloon 402 to retain the delivery wire 100 against the balloon 500. As in the embodiment of FIG. 4, the delivery wire 100 is used to pull the balloon 500 against the LV freewall. As in the embodiments of FIGS. 1-4, the LV ventricular-septal dimension is reduced by anchoring the retention cable in both the septum and LV cavity side of the freewall (FIG. 5C) using coaxial locking mechanism 108.

Those skilled in the art will appreciate that the balloons and coils used as anchoring devices to reestablish more normal LV geometry and function in the exemplary embodiments may be preformed to be concave toward the ventricle so that it will effectively “cup” the left ventricular freewall when pulled against the epicardial surface. Of course, other anchoring mechanisms may be deployed transvenously and/or through a small sub-xiphoid incision or thoracotomy in a manner consistent with contemporary minimally invasive surgical techniques. Also, in all the embodiments, a series of small pads, meshes or balloons may be used to anchor the device against both the infarcted LV wall and the interventricular septum. Such pads, meshes or balloons may also be used to minimize the possibility of fluid leakage as a result of the punctures through the septum and LV wall.

Those skilled in the art will appreciate that the mechanical restraint device described herein may be used to attenuate the effects of chronic post infarct ventricular remodeling and to mitigate ischemic mitral valve regurgitation (IMR) in patients as a result of left ventricular dilation. The device proposed herein offers a minimally invasive way to reduce the septal-lateral dimension of the LV, potentially improving mitral valve function.

Those skilled in the art will also appreciate that the invention may be applied to other applications and may be modified without departing from the scope of the invention. For example, those skilled in the art will appreciate that the devices and techniques of the invention may be used to replace the tricuspid valve as well as the mitral valve. Accordingly, the scope of the invention is not intended to be limited to the exemplary embodiments described above, but only by the appended claims.

What is claimed:

1. A myocardial restraint device comprising:
a restraining wire that is adapted for transvenous delivery to the left ventricle of the heart and then into pericardial space adjacent to an external left ventricular heartwall, a trailing end of said wire forming a tether;
an anchor that holds said restraining wire taut against said external left ventricular heart wall when a force is applied to a proximal end of the restraining wire;
an intraventricular septal anchor that anchors the tether to the ventricular septum; and
a locking mechanism that locks the anchor to the tether,
wherein a distance between the ventricular septum and the ventricular heart wall where the restraining wire is positioned is shortened by tightening the tether connecting the restraining wire to the septal anchor.

2. The device of claim 1, wherein the restraining wire comprises a metal or nitinol wire that is preformed into a spiral coil.

3. The device of claim 2, wherein the restraining wire is preformed to be concave toward the ventricle so as to cup the ventricular heart wall when deployed.

4. The device of claim 2, wherein the restraining wire is preformed to be flat so as to pull in flatter walls of the ventricle when deployed.

5. The device of claim 1, wherein the septal anchor comprises pads, wire meshes, and/or balloons deployed on both sides of the ventricular septum and connected by the tether through the ventricular septum.

6. The device of claim 2, wherein the anchor comprises a distal end of said spiral coil that is recoiled in the pericardial space.

7. The device of claim 1, wherein the anchor comprises a balloon affixed to a distal end of said restraining wire, said balloon adapted for transvenous delivery to the pericardial space and adapted to be inflated in the pericardial space.

8. The device of claim 1, wherein the anchor comprises a restraining coil or mesh adapted to be delivered to said pericardial space through a sub-xiphoid incision or thoracotomy and adapted to anchor a distal end of said restraining wire.

9. The device of claim 1, wherein the anchor comprises a balloon adapted to be delivered to said pericardial space through a sub-xiphoid incision or thoracotomy and adapted to be inflated in the pericardial space so as to anchor a distal end of said restraining wire.

10. A method of deploying a myocardial restraint device in a heart, comprising:
introducing a vascular introducer sheath into a vein and advancing the introducer sheath into a right ventricular cavity of the heart and through the right ventricular cavity to the ventricular septum;
puncturing the ventricular septum with a transeptal needle;
advancing the introducer sheath and a dilator over the transeptal needle into the left ventricular cavity;
puncturing the left ventricular wall at a desired site and advancing the introducer sheath into pericardial space;
removing the dilator and needle;
delivering a restraining wire via the introducer sheath into the pericardial space;
placing an anchoring device on a pericardial side of the left ventricular cavity, said anchoring device adapted to hold said restraining wire taut when a force is applied to a proximal end of said restraining wire;
pulling the restraining wire and anchoring device snug against an epicardial surface of the left ventricular wall;
withdrawing the introducer sheath while maintaining traction on a trailing end of the restraining wire as a tether;
anchoring the tether to a septal anchor placed within the ventricular septum as the introducer sheath is withdrawn from the left ventricle into the right ventricle;
tightening the tether as desired; and
locking the septal anchor to the tether.

11. The method of claim 10, wherein the restraining wire comprises a metal or nitinol wire that is preformed into a spiral coil, and wherein placing an anchoring device on the pericardial side of the left ventricular cavity comprises recoiling a distal end of said spiral coil in the pericardial space.

12. The method of claim 11, further comprising preforming the restraining wire to be concave toward the ventricle so as to cup the ventricular heart wall when deployed.

13. The method of claim 11, further comprising preforming the restraining wire to be flat so as to pull in the posterior wall of the ventricle when deployed.

14. The method of claim 10, wherein placing the anchoring device comprises affixing a balloon to a distal end of said restraining wire, transvenously delivering said balloon to the pericardial space, and inflating the balloon in the pericardial space.

15. The method of claim 10, wherein placing the anchoring device comprises delivering a restraining mesh or coil to said pericardial space through a sub-xiphoid incision or thoracotomy and connecting a distal end of said restraining wire to said restraining mesh or coil.

16. The method of claim 10, wherein placing the anchoring device comprises delivering a balloon to said pericardial space through a sub-xiphoid incision or thoracotomy and inflating the balloon in the pericardial space so as to anchor a distal end of said restraining wire.

17. The method of claim 10, wherein the desired site in the left ventricular wall is at a location of left ventricular dilation or other injury.
